(19) United States
(12) Patent Application Publication
(10) Pub. No.: US 2011/0117566 A1
(43) Pub. Date: May 19, 2011

(54) Model System for Diagnosing Lipid Metabolism

(76) Inventors: Daniel Bernardus Van Schalkwijk, Utrecht (NL); Andreas Freidig, Zeist (NL)

(21) Appl. No.: 12/997,430

(22) PCT Filed: Jun. 10, 2009
(86) PCT No.: PCT/NL2009/050324

§ 371 Date: Jan. 27, 2011

(30) Foreign Application Priority Data
Jun. 10, 2008 (EP) 08157988.0

(51) Int. Cl. G06F 17/10; G06G 7/60; A61B 5/00; C12Q 1/68; C12Q 1/44; G01N 33/92; G01N 33/68; G01N 33/48

(52) U.S. Cl. 435/6; 703/11; 703/2; 600/573; 600/300; 435/19; 436/71; 436/86; 436/94

(57) ABSTRACT

The invention relates to a population model for the analysis of blood lipoprotein physiology in a test subject comprising: a. a submodel for the production of blood lipoproteins; b. a submodel for the lipolysis of blood lipoproteins; c. a submodel for the reabsorption of blood lipoproteins; and d. a submodel relating blood lipoprotein particle size to biochemical composition, more specifically triglyceride content, there-by providing an analysis of the physiological processes underlying a steady state particle population distribution. Each submodel is given as function, using the size of the lipoprotein particle as the independent variable.

### BACKGROUND

The importance of accurately measuring lipid levels in blood is well known. High levels of cholesterol in blood are known to increase the risk of a variety of diseases, such as atherosclerosis and related disorders such as cardiovascular events like stroke and high blood pressure, and metabolic syndrome, comprising a multitude of phenomena, like diabetes. In recent years it has appeared that the constitution of the blood lipids plays an important role in the development of and/or risk of lipid-related disorders.

Lipids are mainly present in the blood in the form of lipoproteins, which are spherical particles that transport cholesterol, triglycerides and other lipids in the bloodstream. Information on the sizes of the lipoprotein constituents is of major importance for a correct diagnosis of lipid-related diseases, but is normally not provided in traditional clinical diagnostics. Currently, several subclasses of lipoproteins are defined: VLDL (very low density lipoprotein) can be divided into 6 subclasses, LDL consists of 4 subclasses (including intermediate-density IDL) and HDL has been divided into 5 subclasses. It has appeared that pathological conditions or the risk thereof relate to the amount and distribution of lipids over these subclasses. Based on these findings an ‘atherogenic lipoprotein phenotype’ has been defined, which takes into account a particle size profile within the LDL class (Austin, M. A. et al., 1990, Circulation 82:495-506). In addition to the cholesterol-based risk factors, apolipoprotein measurements such as the ApoB or the ApoB/ApoA-1 ratio have been found to indicate atherosclerosis risk (Alaupovic, P. 1996, Meth. Enzymol. 263:32-60; Walldius, G. et al., 2006, J. Intern. Med. 259:259-266).

Further improvements in risk assessment can only result from a more detailed understanding of lipoprotein physiology. To increase quantitative insight various multi-compartmental models have been developed to analyze experiments with radioactive or stable isotope labelled lipoprotein constituents. The first models describe the fluxes of apolipoprotein B between lipoprotein fractions (Fisher, W. R. et al., 1980, J. Lipid Res. 21:760-774), with subsequent refinements allowing better data interpretation (Fisher, W. R. et al., 1991, J. Lipid Res. 32:1823-1836; Packard, C. J. et at, 1995, J. Lipid Res. 36:172-187; Maistrom, R. et al., 1997, Arterioscler. Thromb. Vase. Biol. 17:1454-1464). Other models describe the fluxes of triglycerides through the lipoprotein fractions (Harris, W. S. et at, 1990, J. Lipid Res. 31:1549-1558; Barrett, P. H. et at, 1991, J. Lipid Res. 32:743-762; Patterson, B. W. et at, 2002, J. Lipid Res. 43:223-233). Similar models describing other lipoprotein kinetics have also been developed (Campos, H. et al., 2001, J. Lipid Res. 42:1239-1249; Cohn, J. S. et al., 2004, J. Clin. Endocrinol. Metab. 89:3949-3955; Zheng, C. et al., J. Lipid Res. 48:1190-1203). These models were developed to deal with various density-based lipoprotein separation techniques. Now, new measuring techniques such as HPLC and NMR measurements (e.g. U.S. Pat. No. 5,343,389) provide more detailed size-concentration profiles of lipoproteins and their constituents.

The state-of-the art models that have been developed all are based on a so-called compartment model, or reactor model, which is a model in which compartments are defined with their own input and output. There is need for a model that more adequately describes and deals with the biological processes that form the basis of the lipoprotein metabolism.

### SUMMARY OF THE INVENTION

The inventors now have developed a system that is able to reproduce a detailed size-concentration profile of blood lipoproteins and also size classes that correspond to experimental measurements. Examples include the classical VLDL1, VLDL2, IDL and LDL density classes. The model output includes steady state particle concentrations, but can also be applied to analyse fluxes of production, lipolysis and reabsorption at each particle size. The model is calculated deterministically, and specifies how the rate of each of the mentioned fluxes depends on the size of the lipoprotein.

More specifically, the system is presented as a population model for the analysis of blood lipoprotein physiology in a test subject comprising:
a. a submodel for the production of blood lipoproteins;
b. a submodel for the lipolysis of blood lipoproteins;
c. a submodel for the reabsorption of blood lipoproteins; and
d. a submodel relating blood lipoprotein particle size to biochemical composition, more specifically triglyceride content,

thereby providing an analysis of the physiological processes underlying a steady state particle population distribution. In this model each submodel is given as function, using the size of the lipoprotein particle as the independent variable. Preferably, in the submodel for the lipolysis two models are contained, one for extra-hepatic tissue mediated lipolysis and one for hepatic lipolysis. Also preferably, the submodel for the reabsorption is able to distinguish between apoB and apoE mediated reabsorption.

Preferably, the population model according to the invention further comprises one or more of the following submodels:
e. a submodel relating blood lipoprotein particle size to total cholesterol content;
f. a submodel relating blood lipoprotein particle size to free cholesterol content;
g. a submodel relating blood lipoprotein particle size to cholesterol ester content;
h. a submodel relating blood lipoprotein particle size to phospholipid content;
i. a submodel relating blood lipoprotein particle size to total protein content.

In more detail, the population model for the presence of blood lipoproteins in a test subject is a model wherein the total steady-state pool of lipoproteins Qₒᵤₜ in a diameter range [dₐd_{b}] is given by:

$${Q}_{out} ( [ \begin{matrix}{d}_{a} & {d}_{b}\end{matrix} ] ) = {\sum }_{{{d}_{i , j} + \frac{1}{2} {d}_{i , j}^{r} \leq {d}_{b}}_{{d}_{i , j} + \frac{1}{2} {d}_{i , j}^{r} \geq {d}_{a}}}^{ } {Q}_{ss} ( {d}_{i , j} ) + R$$

Where Qₛₛ(d_{i,j}) is the steady state pool calculated in the model at diameter d_{i,j}, and subclass resolution d_{i,j}^{r}. These subclasses have a variable resolution, which is always smaller than 0.01 nm in the current implementation. The subindex i refers to the number of lipolysis steps a particle can maximally have gone through to reach size d and subindex j refers to the subclass number within the size range with that lipolysis step number i. R is the remainder for the boundary subclasses, which partially fall in the selected size range:

$${R}_{low} = \frac{( {d}_{i , j} + \frac{1}{2} {d}_{i , j}^{r} ) - {d}_{a}}{{d}_{i , j}^{r}} {Q}_{ss} ( {d}_{i , j} )$$

$$where {d}_{a} ∈ [ {d}_{i , j} - \frac{1}{2} {d}_{i , j}^{r} , {d}_{i , j} + \frac{1}{2} {d}_{i , j}^{r} ]$$

$${R}_{high} = \frac{{d}_{b} - ( {d}_{i , j} - \frac{1}{2} {d}_{i , j}^{r} )}{{d}_{i , j}^{r}} {Q}_{ss} ( {d}_{i , j} )$$

$$where {d}_{b} ∈ [ {d}_{i , j} - \frac{1}{2} {d}_{i , j}^{r} , {d}_{i , j} + \frac{1}{2} {d}_{i , j}^{r} ]$$

$$R = {R}_{high} + {R}_{low}$$

The steady-state pool in the cascade step Qₛₛ^{d}^{₀}(d_{i,j}) at each size d_{i,j} is given by:

$${Q}_{ss} ( {d}_{i , j} ) = \frac{{J}_{p} ( {d}_{i , j} ) + {J}_{l} ( {d}_{i , j} ) + {J}_{l , liver} ( {d}_{i , j} )}{{k}_{l} ( {d}_{i , j} ) + {k}_{l , liver} ( {d}_{i , j} ) + {k}_{u , liver} ( {d}_{i , j} )}$$

Wherein Jₚ(d_{i,j}) is the particle influx resulting from production, Jₗ(d_{i,j}) is the particle influx resulting from extrahepatic lipolysis, J_{l,liver}(d_{i,j}) is the particle influx resulting from hepatic lipolysis, kₗ is the extrahepatic lipolysis rate, k_{l,liver} is the hepatic lipolysis rate and k_{u,liver} is the particle uptake rate.

Preferably in said model, the influxes due to lipolysis are calculated iteratively as follows:

Jₗ(d_{i,j})=kₗ(d_{i-l,j})·Qₛₛ(d_{i-l,j})

J_{l,liver}(d_{i,j})=k_{l,liver}(d_{i-l,j})·Qₛₛ(d_{i-l,j})

wherein d_{i-l,j} indicates the particle radius before the last lipolysis step

Preferably, in the model of the invention the production of blood lipoproteins into the LDL class is given by the equation

$${J}_{p} ( {d}_{i , j} ) = \frac{{J}_{p , LDL} * ( \begin{matrix}{Φ}_{\overline{{d}_{LDL}} σ} ( {d}_{i , j} + \frac{1}{2} {d}_{i , j}^{r} ) - \\ {Φ}_{\overline{{d}_{LDL}} σ} ( {d}_{i , j} - \frac{1}{2} {d}_{i , j}^{r} )\end{matrix} )}{{Φ}_{\overline{{d}_{LDL}} σ} ( {d}_{LDL max} ) - {Φ}_{\overline{{d}_{LDL}} σ} ( {d}_{LDL min} )}$$

$$for \begin{matrix}{d}_{i , j} + \frac{1}{2} {d}_{i , j}^{r} \leq {d}_{LDL max} \\ {d}_{i , j} - \frac{1}{2} {d}_{i , j}^{r} \geq {d}_{LDL min}\end{matrix}$$

In this equation Jₚ(d_{i,j}) is the influx due to production into a subclass with average particle diameter d_{i,j}, and subclass resolution d_{i,j}^{r}J_{p,LDL} is the production rate in the LDL class, which is fixed based on the production data of each subject. φ is the Gaussian cumulative density function. d̅_̅{̅L̅D̅L̅}̅ stands for the mean diameter of the LDL class, σ is the standard deviation of the distribution curve, subscripts indicate the class to which a diameter refers, and whether it is a minimum or maximum value for that class. In the lower boundary subclass, which lies only partially in the LDL class,

$$( {d}_{i , j} - \frac{1}{2} {d}_{i , j} )$$

is replaced by the lower border of the LDL class, d_{LDLmin}; in the upper boundary subclass

$$( {d}_{i , j} + \frac{1}{2} {d}_{i , j}^{r} )$$

is replaced by the upper border of the LDL class, d_{LDLmax}. For the IDL and VLDL2 classes, the production is defined analogously.

For the VLDL1 class, the normal distribution is replaced by the lognormal distribution as follows:

$${J}_{p} ( {d}_{i , j} ) = \frac{{J}_{p , VLDL 1} * ( {F}_{{μ}_{VLDL 1} , {σ}_{ln}} ( {d}_{i , j} + \frac{1}{2} {d}_{i , j}^{r} ) - {F}_{{μ}_{VLDL 1} , {σ}_{ln}} ( {d}_{i , j} - \frac{1}{2} {d}_{i , j}^{r} ) )}{{F}_{{μ}_{VLDL 1} , {σ}_{ln}} ( {d}_{VLDL 1 max} ) - {F}_{{μ}_{VLDL 1} , {σ}_{ln}} ( {d}_{VLDL 1 min} )}$$

$$for$$

$${d}_{i , j} + \frac{1}{2} {d}_{i , j}^{r} \leq {d}_{VLDL 1 max}$$

$${d}_{i , j} - \frac{1}{2} {d}_{i , j}^{r} \geq {d}_{VLDL 1 min}$$

In this equation F is the lognormal cumulative density function starting at d=d_{VLDL1min} with mean μ_{VLDL1}. In the lower boundary subclass, which lies only partially in the VLDL1 range,

$$( {d}_{i , j} - \frac{1}{2} {d}_{i , j}^{r} )$$

is replaced by d_{VLVL1max}; in the upper boundary subclass

$$( {d}_{i , j} + \frac{1}{2} {d}_{i , j}^{r} )$$

is replaced by d_{VLDL1max}.

Also preferably, in the model of the invention the lipolysis attachment rate in extra-hepatic tissue is given by the formula

$${k}_{l} ( d ) = \{ \begin{matrix}{k}_{l max} ( 1 - exp \frac{- {( d - {d}_{l min} )}^{2}}{2 {σ}_{l}^{2}} ) & for d \geq {d}_{l min} \\ 0 & otherwise\end{matrix}$$

or by

$${k}_{l} ( d ) = \{ \begin{matrix}{k}_{l max} ( 1 - exp ( - {( \frac{d - {d}_{l min}}{\sqrt{{S}_{l}} {σ}_{u , liver}} )}^{{S}_{l}} ) ) & for d \geq {d}_{l min} \\ 0 & otherwise\end{matrix}$$

wherein d is the particle diameter, dₗₘᵢₙ is the minimum size at which lipolysis occurs, kₗₘₐₓ is the maximum lipolysis attachment rate and σₗ and Sₗ are shape parameters.

In a similar way, the lipolysis rate in the liver k_{l,liver} can be given by the formula

k_{l,liver}(d)=k_{a,liver}(d)−k_{u,liver}(d)

wherein the liver attachment rate k_{a,liver} is given either by

$${k}_{a , liver} ( d ) = \{ \begin{matrix}{k}_{a , apoE max} ( \frac{( d - {d}_{a , apoE min} ) exp \frac{- {( d - {d}_{a , apoE min} )}^{2}}{2 {( {σ}_{a , apoE} - {d}_{a , apoE min} )}^{2}}}{( {σ}_{a , apoE} - {d}_{a , apoE min} ) exp ( \frac{- 1}{2} )} ) + {k}_{a , apoB} \\ for d \geq {d}_{a , apoE min} \\ {k}_{a , apoB} \\ for d < {d}_{a , apoE min}\end{matrix}$$

or by

$${k}_{a , liver} ( d ) = \{ \begin{matrix}{k}_{a , apoE max} ( \frac{{f}_{weibullpdf} ( \begin{matrix}d - {d}_{a , apoE min} , \\ {A}_{a , apoE min} , \\ {B}_{a , apoE min}\end{matrix} )}{{f}_{weibulipdf max}} ) + {k}_{a , apoB} & for d \geq {d}_{a , apo} \\ {k}_{a , apoB} & otherw\end{matrix}$$

and the liver uptake rate k_{u,liver} is given by

$${k}_{u , liver} ( d ) = \{ \begin{matrix}( {k}_{a , liver} - {k}_{a , apoB} ) ( 1 - exp \frac{- {( d - {d}_{a , apoE min} )}^{2}}{2 {σ}_{u , liver}^{2}} ) + {k}_{a , apoB} & for d \geq {d}_{a , apoE min} \\ {k}_{a , apoB} & for d < {d}_{a , apoE min}\end{matrix}$$

or by

$${k}_{u , liver} ( d ) = \{ \begin{matrix}( \begin{matrix}{k}_{a , liver} - \\ {k}_{a , apoB}\end{matrix} ) ( \begin{matrix}1 - exp \\ ( - {( \frac{d - {d}_{a , apoE min}}{\sqrt{{S}_{u , liver}} {σ}_{u , liver}} )}^{{S}_{u , liver}} )\end{matrix} ) + {k}_{a , apoB} & for d \geq {d}_{a , apoE min} \\ {k}_{a , apoB} & otherwise\end{matrix}$$

wherein k_{a,apoEmax} is the maximum liver uptake rate due to apoE-mediated reabsorption, k_{a,apoB} is the liver uptake rate due to apoB-mediated reabsorption, d is the particle diameter, d_{a,apoEmin} is the minimum particle diameter at which liver lipolysis takes place and σ_{a,apoE} and σ_{u,liver} are shape parameters and where f_{weibullpdf}(d,A,B) is a weibull probability density function evaluated at d with shape parameters A and B, f_{weibullpdfmax} is the maximum function value attained by this weibull function on the lipoprotein size range from 0 to 200 nm and S_{u,liver} is a shape parameter for the alternative liver uptake function.

In another embodiment, the invention provides a method to determine individual parameters of each submodel using data obtained from a blood sample in a subject comprising;
j. taking a blood sample from said subject;
k. providing a data set from said sample comprising either the number of blood lipoprotein particles in a size class or the chemical composition of said particles, wherein at least 6 size classes are provided;
l. feeding said data to a model according to the invention;
m. finding parameters for the submodels defined in the model of the invention such that the resulting calculated total steady-state pool of lipoproteins Qₛₛ for every diameter d is in agreement with said dataset.

Preferably in said method additionally from said sample or said subject one or more data are provided, including but not limited to data from the group consisting of the ApoC3 content, the ApoA5 content, insulin sensitivity indexes, the sialic acid content, the lipoprotein lipase activity, the hepatic lipase activity, the content of C-reactive protein, the content of adiponectin, gene expression data in blood cells, relevant single nucleotide polymorphisms and copy number variations.

In a further embodiment the invention comprises a method to monitor the development of disease or the effect of a therapy in a patient by performing the method of the invention.

Preferably in the above methods said disease is selected from the group of lipid metabolism disorders, including but not limited to hyper-and hypocholesterolemia, hypertriglyceridemia and hyperlipoproteinemia types I, IIa, IIb, III, IV and V.

In a further embodiment, the invention comprises the use of the method according to the invention to choose a patient specific therapeutic intervention, directed at one or more processes that are described by one or more sub models relating to composition, production lipolysis and reabsorption of lipoproteins.

### LEGENDS TO THE FIGURES

FIG. 1: The model framework consists of three modules. In the Kernel module, a size-structured simulator is used to model the particle population and assess the steady-state model prediction. The first sub-model describes how lipoprotein affinity for the modelled physiological processes depends on particle size. The second sub-model describes how the lipoprotein's biochemical composition depends on particle size. Each module can be modified separately.

FIG. 2: Calculation of change in lipoprotein particle diameter due to a percentage change in triglyceride content. The model calculation proceeds in three steps. First, the model calculates the triglyceride content of the particle at its initial size. Second, it calculates the final triglyceride content after lipolysis, during which the particle loses 52% of its triglycerides. Third, it calculates the particle size corresponding to the calculated final triglyceride content, which is the particle size after lipolysis. The relation between lipoprotein diameter and composition was based on data from Tuzikov et al. (2002, Voprosy Meditsinskoj Khimii, 48:90-91). Solid line—triglyceride mass per particle; striped line—free cholesterol mass per particle; dotted line—cholesteryl ester mass per particle.

FIG. 3: How the rate of each process depends on particle size, based on the fitted parameters shown in Table 1. The processes shown are extrahepatic (LpL-mediated) lipolysis (green), liver attachment (black), liver (HL-mediated) lipolysis (red) and liver uptake (blue). Clear differences between patients are observed.

FIG. 4: Average particle, total cholesterol and triglyceride concentrations of model fits based on flux data of VLDL1, VLDL2, IDL and LDL only. The three lines represent averages of the subjects in the three phenotype groups as determined by Packard et al. (2000, J. Lipid Res. 41:305-318). The striped line indicates phenotype ‘A’ (LDL peak size>26 nm), the solid line phenotype ‘I’ (LDL peak size between 25 and 26 nm) and the dotted line phenotype ‘B’ (LDL peak size<25 nm). Although the data did not contain any particle size information further than the four mentioned classes, the model does reproduce the peak size shift in the LDL size range.

FIG. 5: Total Cholesterol and Triglyceride concentration in model fit for patient 17 (white bars), a simulated lipolysis polymorphism reducing the lipolysis affinity (grey bars) and a simulated apoB-related reabsorption polymorphism, reducing the ApoB-related reabsorption affinity (black bars). The lipolysis polymorphism specifically increases VLDL1 triglycerides. The apoB-related reabsorption polymorphism increases LDL cholesterol. Both simulations are in accordance with observed phenotypes.

FIG. 6: Rate parameter values of two patients estimated based on 6 classes derived from measurements by Liposearch. The patients clearly differ in their individual parameters.

### DETAILED DESCRIPTION OF THE INVENTION

The inventors now are proposing a population model for the distribution of blood lipoproteins in which biological processes like production of lipoproteins, reabsorption of lipoproteins and lipolysis influence the size-dependent distribution of lipoproteins. In this way the steady-state values of the lipoprotein content of a certain size class can be predicted.

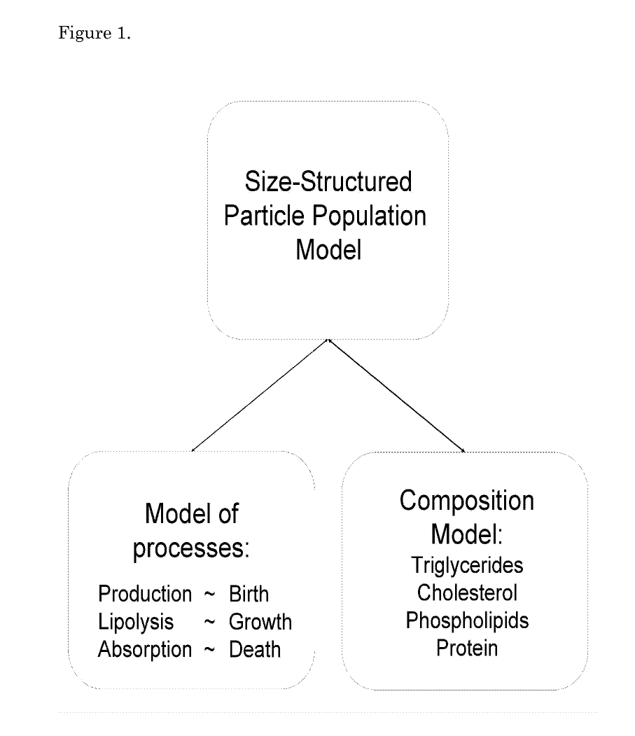

For this, the model consists of a kernel and submodels, shown schematically in FIG. 1. The kernel consists of a size-structured lipoprotein population model which calculates the steady-state lipoprotein levels at different particle sizes. The first submodel (in fact consisting of three submodels) specifies the particle's lifecycle processes of production, lipolysis and reabsorption in a particle-size dependent fashion. The second submodel specifies the relation between particle size and biochemical composition, which is estimated based on empirical data.

Population Model

The kernel of the model framework, the size-structured lipoprotein population model, calculates the steady-state lipoprotein concentration along the size spectrum of ApoB-containing lipoproteins, which ranges from approximately 10 to 100 nm. The steady-state concentration is calculated using information on the production, lipolysis and reabsorption processes affecting single lipoprotein particles from the first submodel. Model calculation proceeds as follows. A particle is produced with a certain diameter, and can go through a variable number of lipolysis steps before being taken up. Each lipolysis step has a corresponding size range which generally becomes smaller as the particles are smaller. A lipolysis step size range is always divided into the same number of subclasses, 1149 in the current implementation (0.01 nm resolution at the crudest). This arrangement makes it possible for all particles that are produced in a particular subclass to flow through to the same subsequent subclass in the lipolysis cascade. In this way the concentration in each particle size range can be calculated efficiently. During the lip olysis cascade, part of the lipoproteins is lost due to reabsorption processes. Once all the processes are specified, the resulting concentration at each cascade step is calculated and associated to the corresponding average particle size. The final particle concentration in the system is found by summing the concentrations of all the overlapping instances of different cascades that fall within a certain class's size region.

After a particle is produced at a given size, its subsequent sizes are determined by the size step due to lipolysis. Using the assumption that the fraction of triglycerides lost f_{tg} is constant at each step, this is given by:

n_{tg}(dᵢ₊₁)=(1−f_{tg})n_{tg}(dₗ)

where n_{tg}(dᵢ) is the initial and n_{tg}(dᵢ₊₁) the final number of triglyceride molecules in a lipoprotein particle, as a function of particle diameter. The corresponding particle diameter is given by the second submodel below. The equation for the total steady-state pool Qₛₛ in a given diameter range [dₐd_{b}] is given by:

$${Q}_{out} ( [ \begin{matrix}{d}_{a} & {d}_{b}\end{matrix} ] ) = {\sum }_{{{d}_{i , j} + \frac{1}{2} {d}_{i , j}^{r} \leq {d}_{b}}_{{d}_{i , j} - \frac{1}{2} {d}_{i , j}^{r} \geq {d}_{a}}}^{ } {Q}_{ss} ( {d}_{i , j} ) + R$$

Where Qₛₛ(d_{i,j}) is the steady state pool calculated in the model at diameter d_{i,j}, and subclass resolution d_{i,j}^{r}. R is the remainder for the boundary subclasses, which partially fall in the selected range:

$${R}_{low} = \frac{( {d}_{i , j} + \frac{1}{2} {d}_{i , j}^{r} ) - {d}_{a}}{{d}_{i , j}^{r}} {Q}_{ss} ( {d}_{i , j} )$$

$$where {d}_{a} ∈ [ {d}_{i , j} - \frac{1}{2} {d}_{i , j}^{r} , {d}_{i , j} + \frac{1}{2} {d}_{i , j}^{r} ]$$

$${R}_{high} = \frac{{d}_{b} - ( {d}_{i , j} - \frac{1}{2} {d}_{i , j}^{r} )}{{d}_{i , j}^{r}} {Q}_{ss} ( {d}_{i , j} )$$

$$where {d}_{b} ∈ [ {d}_{i , j} - \frac{1}{2} {d}_{i , j}^{r} , {d}_{i , j} + \frac{1}{2} {d}_{i , j}^{r} ]$$

$$R = {R}_{high} + {R}_{low}$$

The steady-state pool in the cascade step Qₛₛ^{d}^{ₙ}(dₜ) at each size d_{i,j} is given by:

$${Q}_{ss} ( {d}_{i , j} ) = \frac{{J}_{p} ( {d}_{i , j} ) + {J}_{l} ( {d}_{i , j} ) + {J}_{l , liver} ( {d}_{i , j} )}{{k}_{l} ( {d}_{i , j} ) + {k}_{l , liver} ( {d}_{i , j} ) + {k}_{u , liver} ( {d}_{i , j} )}$$

wherein d_{i,j} is the mean subclass particle diameter in the i-th step of a lipolysis cascade, starting from subclass j within the cascade step size range. Where Jₚ(d_{i,j}) is the particle influx resulting from production, Jₗ(d_{i,j}) is the particle influx resulting from extrahepatic lipolysis, J_{l,liver}(d_{i,j}) is the particle influx resulting from hepatic lipolysis, kₗ is the extrahepatic lipolysis rate, k_{l,liver} is the hepatic lipolysis rate and k_{u,liver} is the particle uptake rate.

Preferably in said model, the influxes due to lipolysis are calculated iteratively as follows:

Jₗ(d_{i,j})=kₗ(d_{i-l,j})·Qₛₛ(d_{i-l,j})

J_{l,liver}(d_{i,j})=k_{l,liver}(d_{i-l,j})·Qₛₛ(d_{i-l,j})

wherein d_{i-l,j} indicates the particle radius before the last lipolysis step.

The model output can be given in various forms. The model can reproduce a detailed size—concentration profile, but also size classes that correspond to experimental measurements. Examples include the classical VLDL1, VLDL2, IDL and LDL density classes, and concentration—size profiles measured by HPLC or NMR (e.g. U.S. Pat. No. 5,343,389). The model output includes steady state particle concentrations, as well as fluxes of production, lipolysis and reabsorption at each particle size.

Process Submodel

The size-dependent models for production, reabsorption and lipolysis are based on biological hypotheses. The hypotheses were translated into mathematical equations. The current model can be can be considered a first functional approximation, to which further biological knowledge can be added in order to arrive at more detailed analysis of lipoprotein physiology.

Production The speed of the production process in each class is based directly on the studied dataset. The size distribution within a size class is based on biological considerations. Production of ApoB-100 VLDL particles is thought to be a two-step process, in which first VLDL2 is produced intercellularly, which can subsequently be fused to a lipid droplet to form VLDL1. This idea was translated to the model by assuming a normal size distribution of secreted particles within the VLDL2, IDL and LDL ranges, since these are expected to vary around a given mean. For the VLDL1 range a lognormal size distribution was assumed, since the size of the fused lipid droplets can vary greatly. The mean of the VLDL 2 fraction was derived from the TG to ApoB ratio of the production in these classes presented by Adiels et al. (2005, J. Lipid Res. 46:58-67). The mean of the VLDL 1 fraction was fitted as a model parameter. Since for IDL and LDL no data were available, the class middle was taken as distribution mean. The standard deviation of the curves was taken as half the distance from the distribution mean to the nearest class border. For the VLDL 1 class this rule applied to the expectation and the square root of the variance of the lognormal production distribution, as specified in the Examples. A correction was applied to ensure all produced particles in each class actually fell inside the specified class.

The production flux into the LDL class can be discretized as follows:

$${J}_{p} ( {d}_{i , j} ) = \frac{{J}_{p , LDL} * ( {Φ}_{\overline{{d}_{LDL}} , σ} ( {d}_{i , j} + \frac{1}{2} {d}_{i , j}^{r} ) - {Φ}_{\overline{{d}_{LDL}} , σ} ( {d}_{i , j} - \frac{1}{2} {d}_{i , j}^{r} ) )}{{Φ}_{\overline{{d}_{LDL}} , σ} ( {d}_{LDL max} ) - {Φ}_{\overline{{d}_{LDL}} , σ} ( {d}_{LDL min} )}$$

$$for$$

$${d}_{i , j} + \frac{1}{2} {d}_{i , j}^{r} \leq {d}_{LDL max}$$

$${d}_{i , j} - \frac{1}{2} {d}_{i , j}^{r} \geq {d}_{LDL min}$$

In this equation Jₚ(d_{i,j}) is the influx due to production into a subclass with average particle diameter d_{i,j}, and subclass resolution d_{i,j}^{r}. The subindex i refers to the number of lipolysis steps a particle can maximally have gone through to reach size d and subindex j refers to the subclass number within the size range with that lipolysis step number i. These subclasses have a variable resolution, which is always smaller than 0.01 nm in the current implementation. J_{p,LDL} is the production rate in the LDL class, which is fixed based on the production data of each subject. φ is the Gaussian cumulative density function. d̅_̅{̅L̅D̅L̅}̅ stands for the mean diameter of the LDL class, σ is the standard deviation of the distribution curve, subscripts indicate the class to which a diameter refers, and whether it is a minimum or maximum value for that class. In the lower boundary subclass, which lies only partially in the LDL class,

$$( {d}_{i , j} - \frac{1}{2} {d}_{i , j}^{r} )$$

is replaced by the lower border of the LDL class, d_{LDLmin}; in the upper boundary subclass

$$( {d}_{i , j} + \frac{1}{2} {d}_{i , j}^{r} )$$

is replaced by the upper border of the LDL class, d_{LDLmax}. For the IDL and VLDL2 classes, the production is defined analogously.

For the VLDL1 class, the normal distribution is replaced by the lognormal distribution as follows:

$${J}_{p} ( {d}_{i , j} ) = \frac{{J}_{p , VLDL 1} * ( {F}_{{μ}_{VLDL 1} , {σ}_{ln}} ( {d}_{i , j} + \frac{1}{2} {d}_{i , j}^{r} ) - {F}_{{μ}_{VLDL 1} , {σ}_{ln}} ( {d}_{i , j} - \frac{1}{2} {d}_{i , j}^{r} ) )}{{F}_{{μ}_{VLDL 1} , {σ}_{ln}} ( {d}_{VLDL 1 max} ) - {F}_{{μ}_{VLDL 1} , {σ}_{ln}} ( {d}_{VLDL 1 min} )}$$

$$for$$

$${d}_{i , j} + \frac{1}{2} {d}_{i , j}^{r} \leq {d}_{VLDL 1 max}$$

$${d}_{i , j} - \frac{1}{2} {d}_{i , j}^{r} \geq {d}_{VLDL 1 min}$$

In this equation F is the lognormal cumulative density function starting at d=d_{VLDL1min} with mean μ_{VLDL1}. In the lower boundary subclass, which lies only partially in the VLDL1 range,

$$( {d}_{i , j} - \frac{1}{2} {d}_{i , j}^{r} )$$

is replaced by d_{VLDL1min}; in the upper boundary subclass

$$( {d}_{i , j} + \frac{1}{2} {d}_{i , j}^{r} )$$

is replaced by d_{VLDL1max}.

The formula for translating the expectation d̅_̅{̅V̅L̅D̅L̅1̅}̅ and standard deviation σ_{VLDL1} for the particle diameter in the VLDL 1 class to the mean (μ) and standard deviation (σᵢₙ) of a lognormal distribution is given by:

$${μ}_{VLDL 1} = ln ( \overline{{d}_{VLDL 1}} - {d}_{VLDL 1 min} ) - \frac{1}{2} ln ( 1 + \frac{{σ}_{VLDL 1}^{2}}{{( \overline{{d}_{VLDL 1}} - {d}_{VLDL 1 min} )}^{2}} )$$

$${σ}_{ln}^{2} = ln ( 1 + \frac{{σ}_{VLDL 1}^{2}}{{( \overline{{d}_{VLDL 1}} - {d}_{VLDL 1 min} )}^{2}} )$$

Values for d̅ and σ in the various production classes can be found in the following table:

| Class | d̅ (nm) | σ (nm) |
|---|---|---|
| LDL | $\frac{25 - {d}_{LDL min}}{2}$ | $\frac{{\overline{d}}_{LDL} - {d}_{LDL min}}{2}$ |
| IDL | 27.5 | 1.25 |
| VLDL2 | 33.54 | 1.77 |
| VLDL1 | 41.87 | 2.94 |

wherein d_{LDLmin} is derived from the free model parameters. It is one lipolysis step smaller than the smallest possible lipolysis attachment size. The mean size of the VLDL2 and VLDL1 classes were derived by comparing the TG to ApoB ratio of the production in these classes presented by Adiels et al. (2005, J. Lipid Res. 46:58-67) to the TG-particle size relation given by Tuzikov et al (2002, Voprosy Meditsinskoj Khimii 48:90-91). Since for IDL and LDL no data are available, the class middle was taken as distribution mean. The standard deviation of the curves was taken as half the distance from the distribution mean to the lower class border.

Extrahepatic tissue—lipolysis In the extrahepatic tissue particles are only lipolysed, reabsorption of particles is negligible (Lichtenstein, L. et al., 2007, Arterioscler. Thromb. Vase. Biol. 27:2420-2427). Lipolysis of lipoproteins in extrahepatic tissue is carried out mainly by lipoprotein lipase (LpL). This enzyme mainly lipolyses larger lipoproteins such as VLDL 1, while VLDL 2 and IDL are lipolyzed to a subsequently lesser extent (Demant, T. et al., 1993, J. Lipid Res. 34:147-156). The particle binds to cell-surface heparin sulphate proteoglycans (HSPG's) mainly through LpL itself, while apoE modulates the binding affinity (de Beer, F. et al., 1999, Arterioscler. Thromb. Vase. Biol. 19:633-637). Multiple LpL's which were already bound to the HSPG's can then be transferred to the lipoprotein, and mediate the lipolysis of the particle. What exactly determines the speed of this lipolysis is not known, although the available surface area, and the biochemical composition (Adiels, M, 2004, PhD thesis, Chalmers Univ. Technol., Göteborg, Sweden), gene expression changes, activators (e.g. ApoCII, ApoE, ApoAV), inhibitors (e.g. ApoCI, ApoCIII, Angpt14) and modulators of LPL expression (e.g. VLDL receptor) of the particle are all hypothesized to influence the speed. In the model framework the lipolysis process is split into two steps: the first step decides whether a particle is bound to a HSPG for lipolysis, the second how many triglycerides it loses during lipolysis. The first step depends on the particle's attachment affinity to HSPG's, which in turn depends an its apolipoprotein composition. This means that the total affinity of the particle for HSPG increases with particle size, until a maximum is reached. Since the exact change in affinity with particle size is not known, this is approximated by a flexible one-parameter function with a shape similar to what would be expected. For this purpose a cumulative density function of the Rayleigh distribution was chosen. The formula for the lipolysis attachment rate in extrahepatic tissue k_{l,ekt}(d) then becomes,

$${k}_{l} ( d ) = \{ \begin{matrix}{k}_{l max} ( 1 - exp \frac{- {( d - {d}_{l min} )}^{2}}{2 {σ}_{l}^{2}} ) & for d \geq {d}_{l min} \\ 0 & otherwise\end{matrix}$$

or alternatively

$${k}_{l} ( d ) = \{ \begin{matrix}{k}_{l max} ( 1 - exp ( - {( \frac{d - {d}_{l min}}{\sqrt{{S}_{l}} {σ}_{u , liver}} )}^{{S}_{l}} ) ) & for d \geq {d}_{l min} \\ 0 & otherwise\end{matrix}$$

where d is the particle diameter, dₗₘᵢₙ is the minimum size at which lipolysis occurs, kₗₘₐₓ is the maximum lipolysis attachment rate and σᵢ and Sₗ are shape parameters. Once a particle is selected for lipolysis, the model assumes that a fixed fraction of the total triglycerides in the particle is lost.

Liver—lipolysis and uptake In the liver the particle first needs to be attached to liver HSPG's. This process is primarily mediated by apoE, so that the attachment does not work for LDL particles without apoE. These LDL particles only contain apoB, which can attach to the LDL receptor and lead to the reabsorption of small particles. Larger particles can either be lipolysed or be reabsorbed. The lipolysis in the liver is primarily mediated by hepatic lipase (HL), an enzyme which functions primarily on smaller apoB and apoE-containing lipoproteins such as IDL, and to a lesser extent on VLDL 2 (Demant, T. et al. 1988, J. Lipid Res. 29:1603-1611). Reabsorption of larger lipoproteins can take place via LRP. Roles for SRB1 and direct incorporation via HSPGs have also been suggested (Out, R. et al., 2004, J. Biol. Chem. 279:18401-18406; MacArthur, J. et al., 2007, J. Clin. Invest. 117:153-164).

In the model liver attachment and further processing are again described as a two-step process. First attachment takes place, mainly mediated by apoE, but with a small contribution from apoB. Although small, this contribution is important especially in the LDL size range, where a small uptake affinity combined with large amounts of particles result in a considerable uptake flux. Since attachment increases and can subsequently decrease with particle size, a Rayleigh probability density function is used to describe this pattern. In order to have its maximum at one, it is scaled using the maximum of this same function, which lies at d=σa,apoE. The liver attachment rate k_{a,liver} can either be given by

$${k}_{a , liver} ( d ) = \{ \begin{matrix}{k}_{a , apoE max} ( \frac{\begin{matrix}( d - {d}_{a , apoE min} ) exp \\ \frac{- {( d - {d}_{a , apoE min} )}^{2}}{2 {( \begin{matrix}{σ}_{a , apoE} - \\ {d}_{a , apoE min}\end{matrix} )}^{2}}\end{matrix}}{( \begin{matrix}{σ}_{a , apoE} - \\ {d}_{a , apoE min}\end{matrix} ) exp ( \frac{- 1}{2} )} ) + {k}_{a , apoB} & for d \geq {d}_{a , apoE min} \\ {k}_{a , apoB} & for d < {d}_{a , apoE min}\end{matrix}$$

or instead by

$${k}_{a , liver} ( d ) = \{ \begin{matrix}{k}_{a , apoE max} ( \frac{{f}_{weibullpdf} ( \begin{matrix}d - {d}_{a , apoE min} , \\ {A}_{a , apoE min} , \\ {B}_{a , apoE min}\end{matrix} )}{{f}_{weibullpdf max}} ) + {k}_{a , apoB} & for d \geq {d}_{a , apoEmin} \\ {k}_{a , apoB} & otherwise\end{matrix}$$

Subsequently, part of the attached lipoproteins are taken up. Since apoB attachment mainly occurs through the LDL receptor all apoB-mediated attachment results in uptake, the part attached through apoE can result in lipolysis. Since HL mainly lipolyses smaller particles the fraction of lipolyzed lipoproteins increases with decreasing particle size. The resulting liver uptake rate k_{u,liver} then becomes.

$${k}_{u , liver} ( d ) = \{ \begin{matrix}( \begin{matrix}{k}_{a , liver} - \\ {k}_{a , apoB}\end{matrix} ) ( 1 - exp \frac{- {( d - {d}_{a , apoE min} )}^{2}}{2 {σ}_{u , liver}^{2}} ) + {k}_{a , apoB} & for d \geq {d}_{a , apoE min} \\ {k}_{a , apoB} & otherwise\end{matrix}$$

An alternative formulation is:

$${k}_{u , liver} ( d ) = \{ \begin{matrix}( \begin{matrix}{k}_{a , liver} - \\ {k}_{a , apoB}\end{matrix} ) ( \begin{matrix}1 - exp \\ ( - {( \frac{d - {d}_{a , apoE min}}{\sqrt{{S}_{u , liver}} {σ}_{u , liver}} )}^{{S}_{u , liver}} )\end{matrix} ) + {k}_{a , apoB} & for d \geq {d}_{a , apoE min} \\ {k}_{a , apoB} & otherwise\end{matrix}$$

The lipolysis rate in the liver k_{l,liver} is then given by:

k_{l,liver}(d)=k_{a,liver}(d)−k_{u,liver}(d)

In these equations k_{a,apoEmax} is the maximum liver uptake rate due to apoE-mediated reabsorption, k_{a,apoB} is the liver uptake rate due to apoB-mediated reabsorption, d is the particle diameter, d_{a,apEmin} is the minimum particle diameter at which liver lipolysis takes place and σ_{a,apoE} and σ_{u,liver} are shape parameters. Furthermore, f_{weibullpdf}(d,A,B) is a weibull probability density function evaluated at d with shape parameters A and B, and f_{weibullpdfmax} is the maximum function value attained by this weibull function on the lipoprotein size range. S_{u,liver} is a shape parameter for the alternative liver uptake function. Note that in these equations the probability functions are not used as such, but rather to indicate the shape of the rate distribution over lipoprotein particle sizes.

Size—Composition Submodel

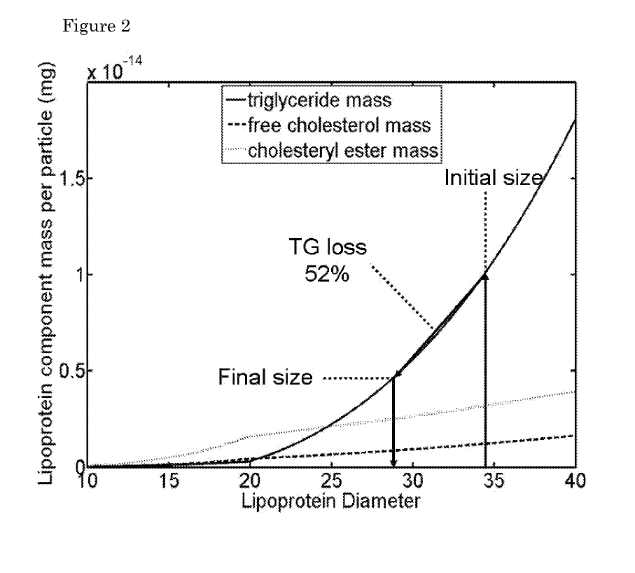

The relation between particle diameter and both particle cholesterol and triglyceride content was based on a dataset presented by Tuzikov et al. (2002, Voprosy Meditsinskoj Khimii 48:90-91). The size—triglyceride relationship was fitted using a sixth degree polynomial, the size-cholesterol relationship by a third-degree polynomial, shown in FIG. 2. Although it is known that the mapping between particle size and biochemical composition is not always one-one, the current approach gives a solid first approximation which may still be improved in future versions of the model. A second option is to use the same model that Tuzikov et al. presented. It is further envisaged, that similar submodels for the relation between particle size and total or free cholesterol content, for particle size and cholesterol ester content, for particle size and phospholipid content and for particle size and total protein content can be developed.

As is shown in the Examples the presently proposed model is capable of predicting the size distribution (population) of blood lipoproteins. The model can also analyse a measured size distribution, and derive parameters that indicate the status of the production, lipolysis and reabsorption processes. In practice the model can calculate this output with minimum input data, as compared to the state-of-the-art models. Clinical data sets are obtained from a blood sample of an individual patient. Each data set should consist of, at least, a size distribution profile of ApoB containing lipoproteins in plasma where the distribution is separated in 6 or more classes and where the size range of each class can be expressed in nm. The distribution profile needs to provide information on either the number of lipoprotein particles per class or on the chemical composition of particles found within the class (such as triglyceride and cholesterol concentration in a class). Advantageously, additional clinical chemical data from the blood sample or from subfractions of the sample can be used to improve the diagnostic parameters of the model, such as the content of ApoC3 and Apo A5, the insulin sensitivity (e.g. HOMA index), the sialic acid content, lipoprotein lipase activity, hepatic lipase activity, C-reactive protein (CRP), adiponectin levels, gene expression data in blood cells and data on genetic background including relevant single-nucleotide polymorphisms (SNP's) and copy number variations.

The model is particularly useful to monitor progress of a disease or a therapy for a disease, where the disease is known to influence the blood lipoprotein distribution and/or content. Such diseases are for instance hypertriglyceridemia, hypercholesteremia and hyperlipoproteinemia types I, IIa, IIb, III, IV and V.

The model is available in an executable computer program, which can be made functional on any personal computer. As such, a digital carrier as computer program product with the program instructions forms part of the present invention. This digital carrier can be a diskette, a hard disk, a memory stick and the like.

Also part of the invention is a computation device (e.g. a computer) that possesses or is provided with the instructions for calculating the population model of the invention. Said computation device will comprise an input section where the data obtained from the clinical sample is introduced. This can be an automatic blood sampling and measuring device which is connected to the computational device and which measures the data from the blood that are needed as input for the model and which then transfers these data to the computational device.

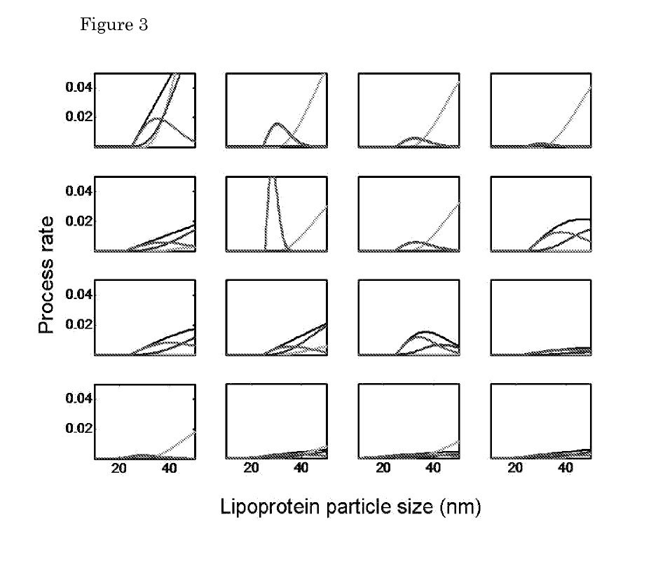

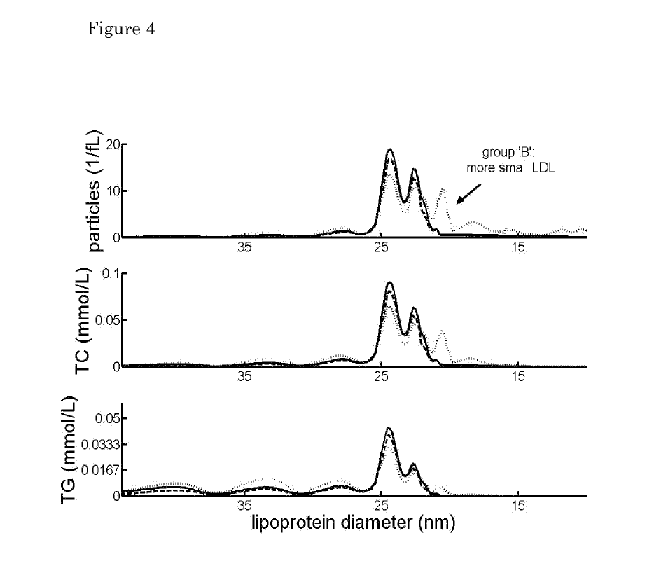

The device further will have an output section that will generate data output, e.g. in the form of graphs as represented in FIGS. 3, 4 and/or 5.

The program needs as input clinical data from a blood sample of an individual patient, as had been specified above, essentially comprising a size distribution of ApoB containing lipoproteins where the distribution is separated in 6 or more classes. Optionally additional clinical data can be entered. Furthermore, of course, specifics about the sample itself and the patient from who it is derived are registered. The output of the program is a graph representing the steady-state distribution of blood lipoproteins and optionally the output can comprise a description of any aberrant physical processes that underlie the resulting information, a proposal for a diagnosis and/or a therapy or a comparison on the severity or progress with any previously obtained results from the same patient. Thus, the model can be of great assistance for the clinician in diagnosis and therapy of patients having a disease relating to changes in the blood lipoprotein profile.

Further, the model can be of use in the study of diseases related to blood lipoproteins and factors affecting the metabolism of blood lipoproteins.

### EXAMPLES

In order to test the model's capability to reproduce measured lipoprotein flux data, the model was fitted to data from a stable isotope labeling study by Packard et al. (2000, J. Lipid Res. 41:305-318).

The individual patient's model outcomes of this study were used as input to the current model. Packard and coworkers divided their subjects into three groups based on the ‘LDL peak size’. Phenotype ‘A’ had an LDL peak size greater than 26 nm, phenotype ‘I’ an LDL peak size between 25 and 26 nm and phenotype ‘B’ an LDL peak size smaller than 25 nm.

In Packard et al. (2000, J. Lipid Res. 41:305-318) the flux data are analyzed by a multi-compartment model to reveal the pool size of each lipoprotein density fraction, as well as the influx from the previous class into the reported class, here interpreted as the lipolysis flux, and the direct catabolism from the fraction, here presented as reabsorption. Also the direct production into each class is quantified. The dataset was considered to be in steady state when the total influx into each category given by production plus lipolysis influx equals the total efflux given by reabsorption plus lipolysis efflux. Datasets in which a large imbalance between total input and total output in one class were found were disregarded, leading to the exclusion of four patients. This selection is necessary because the current model assumes steady-state, which therefore needs to be present in the data. Since the original paper separated the VLDL1, VLDL2, IDL and LDL categories, the model was adapted to reproduce these size classes.

The deviation between the modelled and measured pool sizes is calculated as an average percent difference per datapoint. For the fluxes, this measure is not possible since the data contain several zero entries. Therefore an alternate measure was devised which sums the deviations of all modelled and measured data points, and divides them by the summed flux of the process, also giving a percentage score. Since the model parameters are most sensitive to an accurate pool size fit, the pool size fit was given double the importance of the average fluxes fit. In formula:

$$E = \frac{16}{23} \cdot \frac{{\sum }_{i} \vert {Q}_{i}^{d} - {Q}_{i}^{m} \vert}{{\sum }_{i} {Q}_{i}^{d}} + \frac{3}{23} \frac{{\sum }_{i} \vert {J}_{i}^{l , d} - {J}_{i}^{l , m} \vert}{{\sum }_{i} {J}_{i}^{l , d}} + \frac{4}{23} \frac{{\sum }_{i} \vert {J}_{i}^{u , d} - {J}_{i}^{u , m} \vert}{{\sum }_{i} {J}_{i}^{u , d}}$$

Where E stands for deviation (or error function), Q indicates the pool size, superscript d indicating the data and in the model fit. J stands for a flux, superscripts d and in as before, l indicates lipolysis, u indicates uptake. Subscript i indexes the different data points of each lipolysis size class.

The dataset of Packard et al contains estimations for the apoB in lipoprotein pools of the various classes in mg, and turnover speeds in pools per day. These are converted to particle concentrations and particle fluxes respectively. This needs the assumption that only ApoB-100 is present on lipoprotein particles in the fasted state.

$$n ( \frac{mol}{L} ) = \frac{n ( g )}{{M}_{ApoB \text{-} 100} ( g \text{/} mol ) \cdot {V}_{blood} ( L )}$$

Where n is the number of lipoproteins, M_{ApoB-100} the molar mass of ApoB-100 and V_{blood} the blood volume of a person (taken to be 5 L).

The resulting fitted profile can be viewed in as much detail as is required. This allows the comparison of the modelled ‘LDL peak size’ with the patient's LDL peak size class based on measurements.

The data from the study by Packard et al. (2000, J. Lipid Res. 41:305-318) were fitted using the Levenberg-Marquardt algorithm (implemented in MATLAB version 7.5.0 (R2007b) as the function nlinfit) for optimizing the parameters of the model. The deviation score used is given above. The model was evaluated at sixty-four starting points which were specified by taking two extreme values for each parameter and using a full ‘experimental design’. We then evaluated the middle points in parameter space between 6 points with the lowest error values. In total this results in 79 model evaluations. Of these 79, we used 12 with the lowest error value as starting points for the fitting algorithm. After one fitting round, the 6 best fits were selected as input to a new fitting round; of these outcomes then the three best were selected for a final fitting round. The three final parameter sets were then compared. If the final parameters were found to differ, the whole procedure was repeated using the minima and maxima of each parameter in the set of final parameters as starting points for a new experimental design. If the difference was negligible (parameter difference <1%) the best fitting parameter set was chosen.

It is also possible to inspect the differences in fitted parameters between the groups defined by Packard et al. using the nonparametric Kruskal-Wallis test. This test is suitable for small datasets and does not need the normality assumption a one-way Anova would require. It compares the medians of the parameters describing the three groups of patients.

The fitted model parameters give some information about the processes making up the final lipoprotein profile. To aid interpretation, various process-indicating parameters can be derived from the fitted model parameters:These can either be process indicators, such as the maximum HL activity, the particle size at which it HL affinity is at a maximum and the average apoE-related uptake affinity over the VLDL1 range. They can also be size-class specific indicator parameters of process, age or size averages per particle in that class. For example, the average lipolysis attachment rate per particle in the VLDL1 size class may be calculated. This differs from the ‘transfer from VLDL1 to VLDL2’ variable presented by Packard and coworkers, since it takes into account all lipolysis steps of VLDL1 particles, also those that do not cause the particle to change class.

The model's potential for modeling biological polymorphisms was investigated. Two defects were simulated. The first is a polymorphism in the ApoB-related reabsorption which leads to hypercholesterolemia, the second a defect in LpL lipolysis, which leads to hypertriglyceridemia. Data from Patient “17” in Packard et at (2000, J. Lipid Res. 41:305-318) was chosen for the in-silica experiment. This patient is in the ‘B’ category, with low LDL peak size, and higher CVD risk. The profile of the patient was compared with the same profile firstly if the ApoB-related reabsorption activity was halved, corresponding to an LDL-receptor polymorphism. This was simulated by setting the k_{a,apoB} parameter to half its original value. Secondly the LpL-mediated lipolysis activity was reduced, corresponding to an LpL defect. This was simulated by setting the kₗₘₐₓ value to 50% of its original value. The output of the model is reproduced for the LDL, IDL, VLDL1 and VLDL2 size classes.

Finally, the model was applied to lipoprotein subclass data from a single blood sample. The data were measured by the company Liposearch. The reported cholesterol and triglyceride data at various particle sizes were converted to particle concentrations as follows.

$${V}_{core} = \frac{4 π}{3} {( \overline{d} - {d}_{shell} )}^{3}$$

$${M}_{tg , core} = \frac{\frac{{C}_{tg}}{{ρ}_{tg}}}{\frac{{C}_{tg}}{{ρ}_{tg}} + \frac{{f}_{ce} {C}_{tc}}{{ρ}_{ce}}} {ρ}_{tg} {V}_{core}$$

$${η}_{part} = \frac{{C}_{tg}}{{M}_{tg , core}}$$

wherein V_{core} is the core volume of the lipoprotein particle at average particle size in a class d̅. M_{tg,core} is the triglyceride content of the core in a given class, C_{tg} is the measured triglyceride concentration, ρ_{tg} is the triglyceride density taken to be 0.92 g/cm³, ρ_{ce} is the cholesterol ester density taken to be 0.95 g/cm³ and f_{ce} is the fraction of cholesterol ester versus free cholesterol, based on the biochemical submodel presented above. Finally nₚₐᵣₜ is the number of particles in a specified class. The dimensionality of the data was reduced to six datapoints, by taking together the large VLDL subfractions, the medium and small VLDL subfractions and the very small LDL subfractions and leaving the large medium and small LDL subfractions as reported. The model, using the second option for the liver attachment function, was fitted to this data.

Results

Feasibility of Model Approach

The pool and flux data were well fitted with the model. In all patients the model fit converged to a difference of less then 1% between parameters in the three best fit parameter sets. Table 1 shows the parameters that have been estimated for all subjects from the study by Packard et al. and the corresponding deviations, the definition of which can be found in above. FIG. 3 shows how the processes vary with particle size, given these parameters. The deviation ranged from 1.6% to 16.6% with an average of 7.2%. Only patients 4, 8 and 18 have a deviation above 10%. It is striking that these patients have high uptake in both the LDL and VLDL1 classes, but very low uptake in the intermediate IDL and VLDL2 classes. The current model was not able to reproduce this pattern. Further investigation into the underlying kinetic data could reveal whether this is a physiological phenomenon, or an artifact of Packard's first model analysis of the data. The model analysis shows that the current model could reproduce flux data.

TABLE 1
The fitted model parameter values for 16 subjects from Packard et al. (2000,
J. Lipid Res. 41: 305-318) Only subjects with a data set corresponding to steady-state
were selected. The patients were grouped by Packard et al. into three phenotype
classes, according to their ‘LDL peak size’. Class A had a peak size >26 nm, class I
between 25 and 26 nm and class B <25 nm. Lower LDL peak size is thought to
correspond to a higher risk for cardiovascular disease. The fitted model parameter
average for each of these classes is given, and the significance of inter-group
difference according to the nonparametric Kruskal-Wallis test. An asterisk indicates
the group that differs significantly from the other two groups with p < 0.05.

| | | Liver | Liver | ApoE | ApoB related | Liver | LPL related |
|---|---|---|---|---|---|---|---|
| | | binding min | binding | related | uptake | binding | binding |
| | | size | shape | uptake | affinity | max rate | max rate |
| | % | (nm) | (nm) | shape (nm) | (day⁻¹) | (day⁻¹) | (day⁻¹) |
| Subj. | deviation | d_{a,apoE min} | σ_{a,apoE} | σ_{u,apoE} | k_{u,apoB} | k_{a,apoE max} | k_{l max} |
| 1 | 7.1 | 25.0 | 199.0 | 35.2 | 0.99 | 467.4 | 252.9 |
| 2 | 1.6 | 24.6 | 30.5 | 8953.0 | 0.75 | 22.0 | 131.5 |
| 3 | 4.3 | 25.0 | 32.5 | 4160.9 | 0.45 | 8.0 | 107.4 |
| 4 | 12.1 | 23.7 | 30.3 | 2687.4 | 0.56 | 2.7 | 98.5 |
| 5 | 3.2 | 22.5 | 199.5 | 37.6 | 0.38 | 96.8 | 6.5 |
| 6 | 3.6 | 25.4 | 28.3 | 70.5 | 0.48 | 79.4 | 72.6 |
| 7 | 9.6 | 24.6 | 33.0 | 4641.6 | 0.50 | 8.3 | 78.0 |
| 8 | 16.6 | 25.1 | 47.0 | 41.6 | 0.47 | 30.4 | 0.0 |
| 9 | 4.0 | 24.7 | 63.3 | 42.1 | 0.28 | 28.1 | 0.0 |
| 11 | 7.2 | 24.9 | 199.8 | 36.0 | 0.26 | 126.9 | 13.7 |
| 12 | 9.6 | 25.1 | 36.9 | 39.5 | 0.47 | 21.6 | 0.0 |
| 14 | 3.1 | 23.2 | 51.0 | 48.4 | 0.22 | 6.2 | 0.0 |
| 17 | 7.0 | 21.0 | 29.4 | 4097.2 | 0.38 | 2.9 | 42.9 |
| 18 | 15.9 | 18.6 | 199.6 | 38.3 | 0.29 | 29.6 | 18.9 |
| 19 | 4.1 | 11.0 | 85.6 | 39.2 | 0.24 | 8.1 | 26.6 |
| 20 | 6.3 | 18.8 | 199.8 | 39.2 | 0.29 | 29.2 | 7.4 |

Averages

| | | | | | | |
|---|---|---|---|---|---|---|
| A | 24.5 | 73.7 | 2296.7 | 0.54 | 82.6 | 83.1 |
| I | 24.4 | 95.9 | 41.3 | 0.32 | 51.6 | 4.6 |
| B | (*) 17.3 | 128.6 | 1053.5 | 0.30 | 17.4 | 24.0 |

Significance inter-group difference

| | | | | | | |
|---|---|---|---|---|---|---|
| P | 0.014 | 0.308 | 0.410 | 0.034 | 0.738 | 0.087 |

Prediction of LDL Peak Size Shift

The model could simulate detailed particle size profiles, although it is fitted to pools and fluxes of only four density categories (VLDL 1, VLDL 2, IDL and LDL). These detailed profiles were averaged for all patients in each phenotype class defined by Packard et al. In FIG. 4 these averaged profiles are shown. Although the A and I category profiles overlap, a shift towards lower LDL peak sizes was observed as the phenotype changes from A and I to B, corresponding to the peak size shift measured by Packard. This result points to the physiological realism of the model, since with no size data other than an estimation of the particle size ranges of each density category, the model still reproduced a LDL particle size shift.

The peak size shift was also visible in the model parameters. The Kruskal-Wallis test showed that the median of the lipolysis minimum size significantly decreased from group A and I to group B, with p=0,014. This shows that both the modeled size-concentration profiles and the model parameters qualitatively reproduced the LDL peak size shift between the groups.

Process Identification

Table 2 shows the derived parameters that indicate the status of the various physiological processes. Next to the lipolysis minimum size, also the HL peak attachment rate has significantly different medians between the groups.

Table 3 shows the size-class specific indicator parameters with a significantly changed median between the groups. These include the VLDL1, VLDL2 and LDL average particle age, the IDL and LDL average particle size, the VLDL1, VLDL2 and LDL average lipolysis attachment rate in general and specifically for HL in VLDL1 and LDL, and the LDL uptake rate.

TABLE 2
Derived process indicator parameters for 16 subjects from Packard
et al. (2000, J. Lipid Res. 41: 305-318) Only subjects with a data set
corresponding to steady-state were selected. The patients were grouped
by Packard et al. into three phenotype classes, according to their ‘LDL
peak size’. Class A had a peak size >26 nm, class I between 25 and
26 nm and class B <25 nm. Lower LDL peak size is thought to correspond
to a higher risk for cardiovascular disease. The fitted model parameter
average for each of these classes is given, and the significance of inter-
group difference according to the nonparametric Kruskal-Wallis test.
Significant differences between the groups are seen in the liver attachment
minimum size, which shifts the LDL peak size in the model output. Also
the HL peak attachment rate changes, indicating a change in HL activity.

| | LPL | | | | |
|---|---|---|---|---|---|
| | related | | HL | ApoB | average poE- |
| | binding | HL peak | binding | uptake | related uptake |
| | max rate | binding | peak size | affinity | binding VLDL1 |
| Subj. | (day⁻¹) | rate (day⁻¹) | (nm) | (day⁻¹) | (day⁻¹) |
| 1 | 252.9 | 27.4 | 35.2 | 0.99 | 91.0 |
| 2 | 131.5 | 22.0 | 30.5 | 0.75 | 0.7 |
| 3 | 107.4 | 8.0 | 32.5 | 0.45 | 0.5 |
| 4 | 98.5 | 2.7 | 30.3 | 0.56 | 0.6 |
| 5 | 6.5 | 8.2 | 37.6 | 0.38 | 17.8 |
| 6 | 72.6 | 79.2 | 28.3 | 0.48 | 0.5 |
| 7 | 78.0 | 8.3 | 33.0 | 0.50 | 0.5 |
| 8 | 0.0 | 18.3 | 38.3 | 0.47 | 16.8 |
| 9 | 0.0 | 11.6 | 40.6 | 0.28 | 13.9 |
| 11 | 13.7 | 8.1 | 36.0 | 0.26 | 23.9 |
| 12 | 0.0 | 16.7 | 34.2 | 0.47 | 6.5 |
| 14 | 0.0 | 4.2 | 41.8 | 0.22 | 2.5 |
| 17 | 42.9 | 2.9 | 29.4 | 0.38 | 0.4 |
| 18 | 18.9 | 3.2 | 38.2 | 0.29 | 5.6 |
| 19 | 26.6 | 2.9 | 37.4 | 0.24 | 3.6 |
| 20 | 7.4 | 3.3 | 39.1 | 0.29 | 5.3 |

Averages

| | | | | | |
|---|---|---|---|---|---|
| A | 83.1 | 20.6 | 34.0 | (*) 0.54 | 15.8 |
| I | 4.6 | 9.7 | 37.3 | 0.32 | 11.0 |
| B | 24.0 | (*) 3.1 | 36.0 | 0.30 | 3.7 |

Significance inter-group difference

| | | | | | |
|---|---|---|---|---|---|
| p | 0.087 | 0.043 | 0.512 | 0.034 | 0.539 |

A similar group-comparison analysis was done based on the flux parameters Packard reports in his paper (2000, J. Lipid Res. 41:305-318), and using the same patients as we did. In that case next to the pool sizes, only the transfer rate of VLDL1 to VLDL2 differs significantly between the groups. The model therefore seems to he able to indicate relevant differences in physiology between groups with a differing LDL peak size.

Prediction of Polymorphism Effects

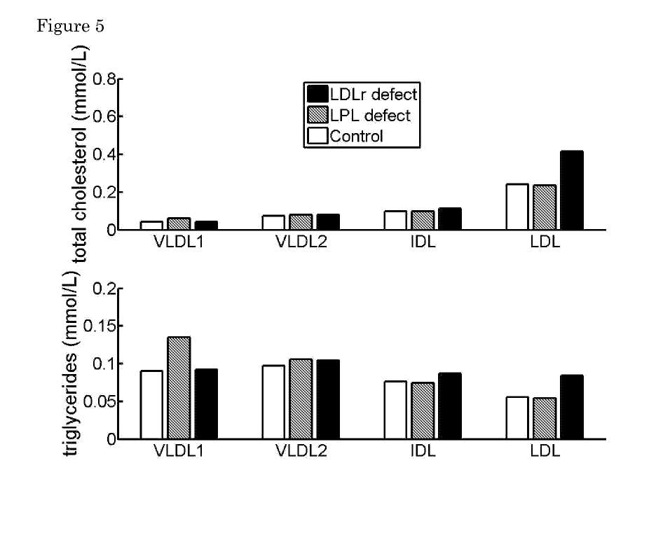

FIG. 5 shows the model fit of patient 17 and simulated polymorphisms affecting ApoB-mediated reabsorption and LpL lipolysis affinity. The cholesterol and triglyceride concentrations in different size classes for the simulated ApoB-mediated reabsorption reduction show the expected hypercholesterolemia (Guerin, M., P. J. et al. 1995. Arterioscler Thromb Vasc Biol 15:1359-1368). The halved ApoB-related reabsorption affinity results in a 1.7-fold increase of the LDL-cholesterol concentration in the blood.

The modelled lipolysis affinity reduction also reproduces the expected hypertriglyceridemia (Okazaki, M., et al. 2005. Arterioscler Thromb Vasc Biol 25:578-584), although less severely than the hypercholesterolemia induced above. Downregulating the LpL lipolysis affinity (by 50%) results in a 1,5 fold increase of VLDL1-triglyceride concentration in the blood. The modelled genetic variants therefore qualitatively resemble the observed phenotype.

Modelling a Single Measurement

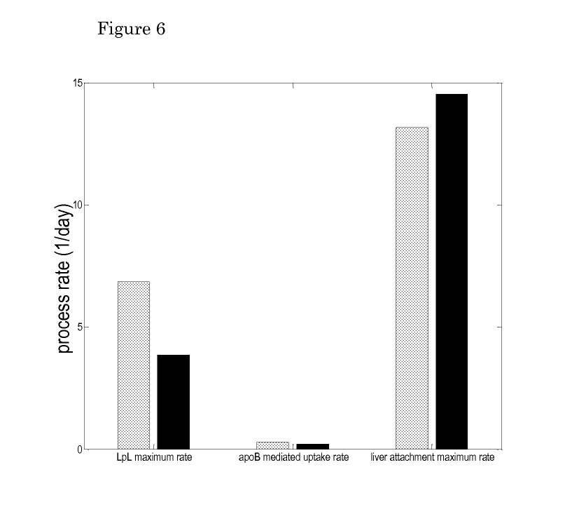

FIG. 6 shows the rate parameter values of two patients estimated based on 6 subclasses as described above. The patients clearly differ in their individual parameters.

TABLE 3
Derived size-specific indicator parameters that showed a significant
difference (p < 0.05) between groups using the nonparametric Kruskal-Wallis test.
Data as in table 2. When we tested the patients we selected using variables from the
original publication this showed a difference between groups in one process - transfer
from VLDL1 to VLDL2. The current analysis showed five significantly different
processes. It indicated lipolysis changes in the LDL, VLDL2 and VLDL1 region, as
well as indicating a changed HL activity in the LDL and VLDL2 range. These changes
were found to be biologically plausible

| | | | | Significance |
|---|---|---|---|---|
| | Group | | | inter-group |
| | means | | | difference |
| units | A | I | B | p-value |

Packard - from published process parameters

| | | | | | |
|---|---|---|---|---|---|
| Transfer from VLDL1 to VLDL2 | (pools/day) | 16.8 | 5.9 | 5.8 | 0.0144 |

Size-specific process indicator parameters

| | | | | | |
|---|---|---|---|---|---|
| Average particle lipolysis rate | day⁻¹ | 0.05 | 0.03 | 0.55 | 0.026 |
| LDL | | | | | |
| Average particle lipolysis rate | day⁻¹ | 13.05 | 8.96 | 3.37 | 0.026 |
| VLDL2 | | | | | |
| Average particle lipolysis rate | day⁻¹ | 25.76 | 7.68 | 7.77 | 0.005 |
| VLDL1 | | | | | |
| Average particle uptake rate | day⁻¹ | 0.54 | 0.32 | 0.32 | 0.042 |
| LDL | | | | | |
| Average particle HL attachment | day⁻¹ | 0.05 | 0.03 | 0.55 | 0.026 |
| rate LDL | | | | | |
| Average particle HL attachment rate | day⁻¹ | 10.94 | 8.85 | 2.77 | 0.034 |
| VLDL2 | | | | | |

Size and age parameters

| | | | | | |
|---|---|---|---|---|---|
| Average particle age LDL | hours | 33.67 | 59.85 | 74.65 | 0.014 |
| Average particle age VLDL2 | hours | 2.16 | 3.96 | 4.91 | 0.026 |
| Average particle age VLDL1 | hours | 0.67 | 1.40 | 1.62 | 0.026 |
| Average particle diameter LDL | nm | 23.45 | 23.52 | 19.22 | 0.027 |
| Average particle diameter IDL | nm | 26.45 | 26.52 | 27.35 | 0.039 |

1. A computer programme product comprising instructions for causing a processor to carry out the steps of calculating a population model for the analysis of blood lipoprotein physiology in a test subject comprising:
a. a submodel for the production of blood lipoproteins;
b. a submodel for the lipolysis of blood lipoproteins;
c. a submodel for the reabsorption of blood lipoproteins; and
d. a submodel relating blood lipoprotein particle size to biochemical composition, more specifically triglyceride content,
wherein each submodel is given as function, using the size of the lipoprotein particle as the independent variable, thereby providing an analysis of the physiological processes underlying a steady state particle population distribution.

2. A computer programme product according to claim 1, wherein in the submodel for the lipolysis two models are contained, one for extra-hepatic tissue mediated lipolysis and one for hepatic lipolysis.

3. A computer programme product according to claim 1-2, wherein the submodel for the reabsorption is able to distinguish between apoB and apoE mediated reabsorption.

4. A computer programme product according to any of claims 1-3, wherein the population model further comprises one or more of the following submodels:
a. a submodel relating blood lipoprotein particle size to total cholesterol content;
b. a submodel relating blood lipoprotein particle size to free cholesterol content;
c. a submodel relating blood lipoprotein particle size to cholesterol ester content;
d. a submodel relating blood lipoprotein particle size to phospholipid content;
e. a submodel relating blood lipoprotein particle size to total protein content.

5. A computer programme product according to claim 1-4 for calculating a population model for the presence of blood lipoproteins in a test subject wherein the total steady-state pool of lipoproteins in a diameter range [dₐd_{b}] is given by:
$${Q}_{out} ( [ {d}_{a} {d}_{b} ] ) = {\sum }_{{{d}_{i , j} + \frac{1}{2} {d}_{i , j}^{r} \leq {d}_{b}}_{{d}_{i , j} - \frac{1}{2} {d}_{i , j}^{r} \geq {d}_{a}}} {Q}_{ss} ( {d}_{i , j} ) + R$$
wherein Qₛₛ(d_{i,j}) is the steady state pool of a cascade step at diameter d_{i,j}, and subclass resolution d_{i,j}^{r}. R is the remainder for the boundary subclasses, which partially fall in the selected range:
$${R}_{low} = \frac{( {d}_{i , j} + \frac{1}{2} {d}_{i , j}^{r} ) - {d}_{a}}{{d}_{i , j}^{r}} {Q}_{ss} ( {d}_{i , j} )$$
$$where$$
$${d}_{a} ∈ [ {d}_{i , j} - \frac{1}{2} {d}_{i , j}^{r} , {d}_{i , j} + \frac{1}{2} {d}_{i , j}^{r} ]$$
$${R}_{high} = \frac{{d}_{b} - ( {d}_{i , j} - \frac{1}{2} {d}_{i , j}^{r} )}{{d}_{i , j}^{r}} {Q}_{ss} ( {d}_{i , j} )$$
$$where$$
$${d}_{b} ∈ [ {d}_{i , j} - \frac{1}{2} {d}_{i , j}^{r} , {d}_{i , j} + \frac{1}{2} {d}_{i , j}^{r} ]$$
$$R = {R}_{high} + {R}_{low}$$

6. A computer programme product according to claim 5, wherein the steady-state pool in the cascade step Qₛₛ^{d}^{₀}(dᵢ) at each size d_{i,j} is given by:
$${Q}_{ss} ( {d}_{i , j} ) = \frac{{J}_{p} ( {d}_{i , j} ) + {J}_{l} ( {d}_{i , j} ) + {J}_{l , liver} ( {d}_{i , j} )}{{k}_{l} ( {d}_{i , j} ) + {k}_{l , liver} ( {d}_{i , j} ) + {k}_{u , liver} ( {d}_{i , j} )}$$
wherein d_{i,j} is the mean subclass particle diameter in the i-th step of a lipolysis cascade, starting from subclass j within the cascade step size range, herein Jₚ(d_{i,j}) is the particle influx resulting from production, Jᵢ(d_{i,j}) is the particle influx resulting from extrahepatic lipolysis, J_{l,liver}(d_{i,j}) is the particle influx resulting from hepatic lipolysis, kₗ is the extrahepatic lipolysis rate, k_{l,liver} is the hepatic lipolysis rate and k_{u,liver} is the particle uptake rate.

7. A computer programme product according to claim 5 or 6, wherein the influx due to extrahepatic lipolysis Jₗ and due to hepatic lipolysis J_{l,liver} at particle diameter d_{i,j} defined as:
J₁(d_{i,j})=kₗ(d_{i-l,j})·Qₛₛ(d_{i-l,j})
J_{l,liver}(d_{i,j})=_i k_{l,liver}(d_{i-l,j})·Qₛₛ(d_{i-l,j})
where kₗis the extrahepatic lipolysis attachment rate, k_{l,liver} is the hepatic lipolysis attachment rate and d_{i-l,j} indicates the particle radius before the last lipolysis step.

8. A computer programme product according to any of claims 5-7, wherein the production of blood lipoproteins in the LDL class is given by the equation
$${J}_{p} ( {d}_{i , j} ) = \frac{{J}_{p , LDL} * ( \begin{matrix}{Φ}_{\overline{{d}_{LDL} ,} σ} ( {d}_{i , j} + \frac{1}{2} {d}_{i , j}^{r} ) - \\ {Φ}_{\overline{{d}_{LDL} ,} σ} ( {d}_{i , j} - \frac{1}{2} {d}_{i , j}^{r} )\end{matrix} )}{\begin{matrix}{Φ}_{\overline{{d}_{LDL} ,} σ} ( {d}_{LDL max} ) - \\ {Φ}_{\overline{{d}_{LDL} ,} σ} ( {d}_{LDL min} )\end{matrix}} for$$
$${d}_{i , j} + \frac{1}{2} {d}_{i , j}^{r} \leq {d}_{LDL max}$$
$${d}_{i , j} - \frac{1}{2} {d}_{i , j}^{r} \geq {d}_{LDL min}$$
wherein in this equation Jₚ(d_{i,j}) is the influx due to production into a subclass with average particle diameter d_{i,j}, and subclass resolution d_{i,j}^{r} wherein the subindices refer to the lipolysis step (i) and the subclass within that lipolysis step range (j), wherein J_{p,LDL} is the production rate in the LDL class, which is fixed based on the production data of each subject, φ is the Gaussian cumulative density function, d̅_̅{̅L̅D̅L̅}̅ stands for the mean diameter of the LDL class, σ is the standard deviation of the distribution curve, and wherein subscripts indicate the class to which a diameter refers, and whether it is a minimum or maximum value for that class, and wherein in the lower boundary subclass, which lies only partially in the LDL class,
$$( {d}_{i , j} - \frac{1}{2} {d}_{i , j}^{r} )$$
is replaced by the lower border of the LDL class, d_{LDLmin}; wherein in the upper boundary subclass
$$( {d}_{i , j} + \frac{1}{2} {d}_{i , j}^{r} )$$
is replaced by the upper border of the LDL class, d_{LDLmax}.

9. A computer programme product according to claim 8, wherein for the VLDL1 class, the normal distribution is replaced by the lognormal distribution as follows:
$${J}_{p} ( {d}_{i , j} ) = \frac{{J}_{p , VLDL 1} * ( \begin{matrix}{F}_{{μ}_{VLDL 1} , {σ}_{ln}} ( {d}_{i , j} + \frac{1}{2} {d}_{i , j}^{r} ) - \\ {F}_{{μ}_{VLDL 1} , {σ}_{ln}} ( {d}_{i , j} - \frac{1}{2} {d}_{i , j}^{r} )\end{matrix} )}{\begin{matrix}{F}_{{μ}_{VLDL 1} , {σ}_{ln}} ( {d}_{VLDL 1 max} ) - \\ {F}_{{μ}_{VLDL 1} , {σ}_{ln}} ( {d}_{VLDL 1 min} )\end{matrix}} for$$
$${d}_{i , j} + \frac{1}{2} {d}_{i , j}^{r} \leq {d}_{VLDL 1 max}$$
$${d}_{i , j} - \frac{1}{2} {d}_{i , j}^{r} \geq {d}_{VLDL 1 min}$$
wherein F is the lognormal cumulative density function starting at d=d_{VLDL1min} with mean μ_{VLDL1} and wherein in the lower boundary subclass, which lies only partially in the VLDL1 range,
$$( {d}_{i , j} - \frac{1}{2} {d}_{i , j}^{r} )$$
is replaced by d_{VLDL1min}; and wherein in the upper boundary subclass
$$( {d}_{i , j} + \frac{1}{2} {d}_{i , j}^{r} )$$
is replaced by d_{VLDL1max}.

10. A computer programme product according to any of claims 5-9, wherein the lipolysis attachment rate in extra-hepatic tissue is given by the formula
$${k}_{l} ( d ) = \{ \begin{matrix}{k}_{l max} ( 1 - exp \frac{- {( d - {d}_{l min} )}^{2}}{2 {σ}_{l}^{2}} ) & for d \geq {d}_{l min} \\ 0 & otherwise\end{matrix}$$
or alternatively
$${k}_{l} ( d ) = \{ \begin{matrix}{k}_{l max} ( 1 - exp ( - {( \frac{d - {d}_{l min}}{\sqrt{{S}_{l}} {σ}_{u , liver}} )}^{{S}_{l}} ) ) & for d \geq {d}_{l min} \\ 0 & otherwise\end{matrix}$$
wherein d is the particle diameter, dₗₘᵢₙ is the minimum size at which lipolysis occurs, kₗₘₐₓ is the maximum lipolysis attachment rate and σₗ and Sₗ are shape parameters.

11. A computer programme product according to any of claims 5-10, wherein the lipolysis rate in the liver k_{l,liver} is given by the formula
k_{l,liver}(d)=k_{a,liver}(d)−k_{u,liver}(d)
wherein the liver attachment rate k_{a,liver} is given either by
$${k}_{a , liver} ( d ) = \{ \begin{matrix}{k}_{a , apoE max} ( \frac{\begin{matrix}( d - {d}_{a , apoE min} ) \\ exp \frac{- {( d - {d}_{a , apoE min} )}^{2}}{2 {( {σ}_{a , apoE } - {d}_{a , apoE min} )}^{2}}\end{matrix}}{\begin{matrix}( {σ}_{a , apoE } - {d}_{a , apoE min} ) \\ exp \frac{- {( {σ}_{a , apoE} - {d}_{a , apoE min} )}^{2}}{2 {( {σ}_{a , apoE } - {d}_{a , apoE min} )}^{2}}\end{matrix}} ) + {k}_{a , apoB} & for d \geq {d}_{a , apoE n} \\ {k}_{a , apoB} & otherwise\end{matrix}$$
or by
$${k}_{a , liver} ( d ) = \{ \begin{matrix}{k}_{a , apoE max} ( \frac{{f}_{wetbullpdf} ( \begin{matrix}d - {d}_{a , apoE min} , \\ {A}_{a , apoE min} , {B}_{a , apoE min}\end{matrix} )}{{f}_{wetbullpdf max}} ) + {k}_{a , apoB} & for d \geq {d}_{a , apoE min} \\ {k}_{a , apoB} & otherwise\end{matrix}$$
and the liver uptake rate k_{u,liver} is given by
$${k}_{u , liver} ( d ) = \{ \begin{matrix}\begin{matrix}( {k}_{a , liver} - {k}_{a , apoB} ) \\ ( 1 - exp \frac{- {( d - {d}_{a , apoE min} )}^{2}}{2 {σ}_{u , liver}^{2}} ) + {k}_{a , apoB}\end{matrix} & for d \geq {d}_{a , apoE m} \\ {k}_{a , apoB} & otherwise\end{matrix}$$
or by
$${k}_{u , liver} ( d ) = \{ \begin{matrix}\begin{matrix}( {k}_{a , liver} - {k}_{a , apoB} ) \\ ( 1 - exp ( - {( \frac{d - {d}_{a , apoE min}}{\sqrt{{S}_{u , liver}} {σ}_{u , liver}} )}^{{S}_{u , liver}} ) ) + {k}_{a , apoB}\end{matrix} & for d \geq {d}_{a , apoE min} \\ {k}_{a , apoB} & otherwise\end{matrix}$$
wherein k_{a,apoEmax} is the maximum liver uptake rate due to apoE-mediated reabsorption, k_{a,apoB} is the liver uptake rate due to apoB-mediated reabsorption, d is the particle diameter, d_{a,apoEmin} is the minimum particle diameter at which liver lipolysis takes place and σ_{a,apoE} and σ_{u,liver} are shape parameters and where f_{weibullpdf}(d,A,B) is a weibull probability density function evaluated at d with shape parameters A and B, f_{weibullpdfmax} is the maximum function value attained by this weibull function on the lipoprotein size range from 0 to 200 nm, and S_{u,liver} is a shape parameter.

12. Method to determine individual parameters in each submodel of a population model for the analysis of blood lipoprotein physiology in a test subject using data obtained from a blood sample in said subject comprising:
a. taking a blood sample from said subject;
b. providing a data set from said sample comprising either the number of blood lipoprotein particles in a size class or the chemical composition of said particles, wherein at least 6 size classes within the range of ApoB-containing particles are provided;
c. feeding said data to a model as defined in claims 1-11;
d. finding parameters for the submodels defined in claim 1-11 such that the resulting calculated total steady-state pool of lipoproteins Qₛₛ for every diameter d is in agreement with said dataset.

13. Method according to claim 12 wherein additionally from said sample or said subject one or more data are provided, including but not limited to data from the group consisting of the ApoC3 content, the ApoA5 content, insulin sensitivity indexes, the sialic acid content, the lipoprotein lipase activity, the hepatic lipase activity, the content of C-reactive protein, the content of adiponectin, gene expression data in blood cells, relevant single nucleotide polymorphisms and copy number variations.

14. Method to monitor the development of disease or the effect of a therapy in a patient by performing the method of claim 12 or 13.

15. Method according to any of claims 12-14, wherein said disease is selected from the group of lipid metabolism disorders, including but not limited to hyper-and hypocholesterolemia, hypertriglyceridemia and hyperlipoproteinemia types I, IIa, IIb, III, IV and V.

16. Use the method of any of claims 12-15 to choose a patient specific therapeutic intervention, directed at one or more processes that are described by one or more sub models relating to composition, production lipolysis and reabsorption of lipoproteins.

17. A computational device equipped with instructions for causing the processor of said computational device to carry out the steps of calculating a population model as defined in any of claims 1-11.
